(19) United States
(12) Patent Application Publication
(10) Pub. No.: US 2012/0196867 A1
(43) Pub. Date: Aug. 2, 2012

(54) COMPOSITION COMPRISING A PDE4 INHIBITOR AND A PDE5 INHIBITOR

(75) Inventors: Torsten DUNKERN, Stockach (DE); Armin HATZELMANN, Konstanz (DE); Christian SCHUDT, Konstanz (DE); Friedrich GRIMMINGER, Butzbach (DE); Hossein Ardeschir GHOFRANI, Gießen (DE)

(73) Assignee: NYCOMED GMBH, Konstanz (DE)

(21) Appl. No.: 13/445,675

(22) Filed: Apr. 12, 2012

Related U.S. Application Data
(63) Continuation of application No. 12/785,973, filed on May 24, 2010.
Application No. 12/785,973 is a continuation of application No. 10/556,888, filed on Nov. 15, 2005, now abandoned.

(30) Foreign Application Priority Data
May 22, 2003 (EP) 03011609.9

(51) Int. Cl. A61K 31/53; A61K 31/497; A61P 11/00; A61K 31/4985

(52) U.S. Cl. 514/243; 514/250; 514/252.16

(57) ABSTRACT

The claimed subject matter relates to pharmaceutical compositions comprising a PDE4 inhibitor and a PDE5 inhibitor and the treatment of a disease in which phosphodiesterase 4 (PDE4) and/or phosphodiesterase 5 (PDE5) is detrimental.

### FIELD OF APPLICATION OF THE INVENTION

The invention relates to the combination of certain known active compounds for therapeutic purposes. The substances used in the combination according to the invention are known active compounds from the phosphodiesterase 4 (PDE4) inhibitor class and known active compounds from the phosphodiesterase 5 (PDE5) inhibitor class. Their combined use in the sense according to the invention for therapeutic purposes has not yet been described in prior art.

### PRIOR ART

In the healthy lung of humans both at rest and during exercise there are always areas of good and poor or absolutely no ventilation existing simultaneously side by side (ventilation in homogeneity). An as yet unknown mechanism ensures that there is little or no perfusion of the capillaries adjacent to alveoli with little or no ventilation. This occurs in order to minimize inefficient perfusion of areas of the lung which are not involved in gas exchange. During bodily exercise, the distribution of ventilation changes (recruitment of new alveoli) and there is increased perfusion of the relevant capillary bed. Conversely, when there is less ventilation due to physiological or pathological processes (airway obstruction), the capillary flow is reduced through vasoconstriction. This process is referred to as “hypoxic vasoconstriction” (Euler-Liljestrand mechanism). When this adaptation mechanism is impaired (“mismatch”), there may, despite adequate ventilation and normal perfusion of the lungs, be a more or less pronounced collapse of the gas exchange function, which can be compensated only inadequately despite a further increase in ventilation or perfusion. Under these conditions there are regions which are not ventilated but are well perfused (shunt flow perfusion) and those which are well ventilated but not perfused (dead space ventilation). The consequences of this ventilation/perfusion mismatch are hypoxaemia (deterioration in gas exchange with an associated decrease in the arterial oxygen saturation), wasted perfusion (uneconomical perfusion of unventilated areas) and wasted ventilation (uneconomical ventilation of poorly perfused areas).

In patients with inflammatory and degenerative lung disorders such as, for example, chronic obstructive pulmonary disease (COPD), bronchitis, bronchial asthma, pulmonary fibroses, emphysema, interstitial pulmonary disorders and pneumonias there is observed to be partial or global respiratory failure. The cause is inadequate adaptation of the intrapulmonary perfusion conditions to the inhomogeneous pattern of the distribution of ventilation. The mismatch derives from the effect of vasoactive (inflammatory) mediators which prevail over the physiological adaptation mechanism and in part from structural changes of the lung capillaries which develop during disease progression. This effect is particularly evident during exercise and when the oxygen demand is increased and it is manifested by dyspnoea (hypoxia) and limitation of performance.

Chronic Obstructive Pulmonary Disease (COPD) is a major public health problem projected to rank fifth in 2020 as a worldwide burden of disease according to a study published by the World Bank/World Health Organization [Murray C J L, Lopez A D. Evidence-based health policy—lessons from the global burden of disease study. Science 1996; 274:740-3]. COPD is a disease state characterized by airflow limitation that is not fully reversible, also upon treatment with bronchodilators. The airflow limitation is usually both progressive and associated with an abnormal inflammatory response of the lungs to noxious particles or gases (e.g. cigarette smoke). COPD is characterized by chronic inflammation throughout the airways, parenchyma, and pulmonary vasculature. This inflammation mechanistically clearly differs from that of asthma, which might explain the limited benefit of corticosteroid treatment in stable disease management of those patients. In addition, other processes are thought to be important in the pathogenesis of COPD, i.e. structural changes/remodelling of the airways as well as of the pulmonary capillaries leading to reduced blood perfusion and an endothelial dysfunction. Up to now a curative therapy for COPD is not available. In use are anti-cholinergic drugs (ipratropium bromide, tiotropium bromide and oxitropium bromide) and short- and long-acting β-adrenoreceptor-agonists (salmeterol, terbutalin-sulphate). β2-agonist-treatment can be associated with several side-effects such as tachycardia, unrest felling, sleep disturbances and tremor. Observed side-effects of tiotropium bromide and ipratropium bromide treatment are the development of a dry mouth, tremor, tachykardia and obstipation. Anti-inflammatory acting corticosteroids are not fully established for COPD treatment, since their effects in the daily management of stable COPD are very small, provide only symptomatic relief, do not improve survival and side-effects (e.g. development of sooroesophagitis) have to be considered.

Bronchial asthma is a widespread chronic inflammatory disease affecting worldwide 5% of adults and 5-15% of all children. In the U.S. 14 million people are suffering from asthma of which each year 500,000 are hospitalized and more than 5,000 die as the clinical endpoint of asthma. In asthma inflammation leads to airflow limitation in the lung because of acute bronchoconstriction, swelling of the airway wall, chronic mucus plug formation and airway wall remodelling. Structurally large and small airways are filled with plugs comprised of a mixture of mucus, serum proteins, inflammatory cells and cell debris. Besides the infiltration with eosinophile granulocytes and lymphocytes, a hypertrophy of epithelial goblet and smooth muscle cells, a microvascular leakage, a epithelial disrupture and a basement membrane thickening can be observed. During status asthmaticus a strong ventilation/perfusion mismatch can be observed. In therapy short- and long-acting β2-agonists are used. However undesired side-effects are described including tachycardia, unrest felling, sleep disturbances and tremor. Chromoglycate (DNCG) is often used for the treatment of children. Side-effects mainly described for DNCG are bronchospasms, cough, gastrointestinal side-effects and dermatitis. Depending on the asthma patient corticosteroids are used via the inhalative, oral and intra-venous (during status asthmaticus) route of administration. Depending on the route of administration side-effects might occur such as osteomalacy, the development of the Cushing-syndrome, depression, adipositas, induction of diabetes mellitus and an enhanced susceptibility for infections.

Rheumatoid arthritis (RA) is the most common rheumatic diseases affecting 0.5% of all people in Germany and about 2.1 million people in the USA. Features of RA are bilateral tender, warm, swollen joints, joint inflammation, fatigue, occasional fever, long-lasting pain and stiffness in the morning. In RA the immune system attacks cells within the joint capsule leading to an autoimmune inflammation called synovitis. In addition to the local inflammation of the joints rheumatoid patients exhibit an increased frequency of cardiovascular disease caused by an associated vasculitis [Bacon, P. A. et al. The role of endothelial cell dysfunction in the cardiovascular mortality of RA. Int. Rev. Immunol. 2002, 21(1): 1-17]. Endothelial dysfunction causes changes in endothelial dependent vasodilatation [Hurlimann, D. et al. Anti-tumor necrosis factor-alpha treatment improves endothelial function in patients with rheumatoid arthritis Circulation 2002; 106(17): 2184-2187]. Medications commonly used to treat RA provide relief from pain and inflammation. Reduction of pain, swelling and inflammation is reached by treatment with analgesics (e.g. acetaminophen) and nonsteroidal anti-inflammatory drugs (NSAIDs, e.g. ibuprofen, celecoxib and rofecoxib). To alter the course of the disease disease-modifying antirheumatic drugs (DMARDs) are used (e.g. gold (Myochrysine), antimalarials (Plaquenil), penicillamine (Depen)). Corticosteroids such as prednisone and methylprednisolone are also used because of their anti-inflammatory and immunosuppressive effects.

The 3′,5′-cyclic nucleotide phosphodiesterases (PDEs) comprise a large class of enzymes divided into at least eleven different families which are structurally, biochemically and pharmacologically distinct from one another.

PDE4s are characterized by selective, high affinity hydrolytic degradation of the second messenger cyclic nucleotide, adenosine 3′,5′-cyclic monophosphate (cAMP). A number of selective and potent inhibitors of the PDE4s have been discovered in recent years for treatment of COPD or Asthma.

Known PDE4 inhibitors are reviewed in Dal Piaz et al. [Dal Piaz V et al. (2000) European Journal of Medicinal Chemistry 35: 463] and Wolda [Wolda S L. PDE4 Inhibitors and chronic obstructive pulmnonary disease. Emerging Drugs, Ashley Publications, London, GB, Vol. 5, No. 3, 2000, pages 309-319].

Compton et al. (Comptom C H et al. (2001) Lancet 358: 265) discloses the effectiveness of maintenance treatment of COPD patients with the PDE4 inhibitor Cilomilast.

PDE5s are characterized by selective, high affinity hydrolytic degradation of the second messenger cyclic nucleotide, guanosine 3′,5′-cydic monophosphate (cGMP). A whole series of PDE5 inhibiting substances are known from the prior art and are described as potent and effective substances for the treatment of erectile dysfunction and pulmonary hypertension.

Ghofrani et al. (Ghofrani H A et al. (2002) Lancet 360: 895) disclose effects of a Sildenafil therapy on patients with lung fibrosis and pulmonary hypertension. Ghofrani et al. demonstrates that Sildenafil acts selectively in well-ventilated areas of the lung by improving the gas exchange.

McPhershon et al. (McPhershon et al. (1999) FEBS Letters 464: 48) demonstrate that a PDE5-inhibitor restores the defect in isoproterenol induced mucine secretion in cells, which resemble the phenotype of cells involved in cystic fibrosis.

Torphy et al. (Torphy T J at al. (2000) Trends in Pharmacological Sciences 21: 157) discloses in a meeting report that the action of PDE4-inhibitors in COPD and RA is due to their well-known suppression of the release of inflammatory mediators, as TNF and IL-12 from various inflammatory cells (neutrophils, macrophages). In the same report, Torphy et al. describes the use of PDE5-inhibitors for the treatment of erectile dysfunction.

### SUMMARY OF THE INVENTION

The invention relates to pharmaceutical compositions comprising a PDE4 inhibitor in combination with a PDE5 inhibitor and methods for preventing or reducing the onset of symptoms of a disease in which phosphodiesterase 4 (PDE4) and/or phosphodiesterase 5 (PDE5) activity is detrimental, and methods for treating or reducing the severity of a disease in which phosphodiesterase 4 (PDE4) and/or phosphodiesterase 5 (PDE5) activity is detrimental.

In particular it relates to compositions and methods for treating a disease mediated by phosphodiesterase 4 (PDE4) and/or phosphodiesterase 5 (PDE5) activity by administering a PDE4 inhibitor in combination with a PDE5 inhibitor.

In this connection, it is the object of the present invention to make available a certain therapeutic, which fulfills the following conditions:
Pronounced anti-inflammatory action and
Rematching effect and/or anti-remodelling effect.

According to present invention, the term “rematching effect” refers to the ability of the combined use of a PDE4 inhibitor and a PDE5 inhibitor to dilate vessels in the pulmonary circulation and, at the same time, to redistribute the blood flow within the lung in favour of the well-ventilated areas of the lung. Thereby the disease associated shunt perfusion within the lung is reduced. Rematching leads to an improvement in the gas exchange function both at rest and during physical exercise and thereby to an improvement in arterial oxygen saturation.

According to present invention, the term “anti-remodelling effect” refers to the ability of the combined use of a PDE4 inhibitor and a PDE5 inhibitor to restore the impaired balance between proliferation and cell death of smooth muscle cells, fibroblasts and epithelial cells and/or to reduce excessive production of extracellular matrix in the vasculature of the lung.

It has now been found that the combined use of a PDE4 inhibitor and a PDE5 inhibitor fulfills the above mentioned conditions.

Accordingly, the invention relates in a first aspect to the combined use of a PDE4 inhibitor and a PDE5 inhibitor for preventing or reducing the onset of symptoms of a disease, or treating or reducing the severity of a disease in a patient in need thereof, in which disease phosphodiesterase 4 (PDE4) and/or phosphodiesterase 5 (PDE5) activity is detrimental.

In another aspect of present invention, there is provided the use of a combination of a PDE4 inhibitor and a PDE5 inhibitor for the preparation of a medicament for preventing or reducing the onset of symptoms of a disease, or treating or reducing the severity of a disease in a patient in need thereof, in which disease phosphodiesterase 4 (PDE4) and/or phosphodiesterase 5 (PDE5) activity is detrimental.

In another aspect of present invention, there is provided a method for preventing or reducing the onset of symptoms of a disease in which phosphodiesterase 4 (PDE4) and/or phosphodiesterase 5 (PDE5) activity is detrimental, or treating or reducing the severity of a disease in which phosphodiesterase 4 (PDE4) and/or phosphodiesterase 5 (PDE5) activity is detrimental by administering to a patient in need thereof a fixed combination of an effective amount of a PDE4 inhibitor and a PDE5 inhibitor, and optionally a pharmaceutically acceptable carrier.

In another aspect of present invention, there is provided a method for preventing or reducing the onset of symptoms of a disease in which phosphodiesterase 4 (PDE4) and/or phosphodiesterase 5 (PDE5) activity is detrimental, or treating or reducing the severity of a disease in which phosphodiesterase 4 (PDE4) and/or phosphodiesterase 5 (PDE5) activity is detrimental by administering to a patient in need thereof a free combination of an effective amount of a PDE4 inhibitor and optionally a pharmaceutically acceptable carrier and a PDE5 inhibitor and optionally a pharmaceutically acceptable carrier.

In another aspect of this invention, there is provided a method for preventing or reducing the onset of symptoms of a disease in which phosphodiesterase 4 (PDE4) and/or phosphodiesterase 5 (PDE5) activity is detrimental, or treating or reducing the severity of a disease in which phosphodiesterase 4 (PDE4) and/or phosphodiesterase 5 (PDE5) activity is detrimental by administering to a patient in need thereof simultaneously an effective amount of (1) a PDE4 inhibitor and (2) a PDE5 inhibitor.

In another aspect the invention relates to a method for preventing or reducing the onset of symptoms of a disease in which phosphodiesterase 4 (PDE4) and/or phosphodiesterase 5 (PDE5) activity is detrimental, or treating or reducing the severity of a disease in which phosphodiesterase 4 (PDE4) and/or phosphodiesterase 5 (PDE5) activity is detrimental by administering to a patient in need thereof in succession, close in time or remote in time, in any order whatever to a patient in need thereof an effective amount of (1) a PDE4 inhibitor and (2) a PDE5 inhibitor.

In another aspect the invention relates to a method for preparing a pharmaceutical composition which is effective for preventing or reducing the onset of symptoms of a disease in which phosphodiesterase 4 (PDE4) and/or phosphodiesterase 5 (PDE5) activity is detrimental, or treating or reducing the severity of a disease in which phosphodiesterase 4 (PDE4) and/or phosphodiesterase 5 (PDE5) activity is detrimental, which method comprises mixing an effective amount of a PDE4 inhibitor and a PDE5 inhibitor with a pharmaceutically acceptable carrier.

In another aspect the invention relates to a pharmaceutical composition comprising as a fixed combination an effective amount of a PDE4 inhibitor and an effective amount of a PDE5 inhibitor, and optionally a pharmaceutically acceptable carrier.

In another aspect the invention relates to a pharmaceutical composition comprising as a fixed oral combination an effective amount of a PDE4 inhibitor and an effective amount of a PDE5 inhibitor, and optionally a pharmaceutically acceptable carrier.

In another aspect the invention relates to a pharmaceutical composition comprising as a free combination (a) an effective amount of a PDE4 inhibitor and optionally a pharmaceutically acceptable carrier and (b) an effective amount of a PDE5 inhibitor and optionally a pharmaceutically acceptable carrier.

In another aspect of present invention there is provided the use of a pharmaceutical composition comprising as a fixed combination an effective amount of a PDE4 inhibitor and an effective amount of a PDE5 inhibitor, and optionally a pharmaceutically acceptable carrier for the treatment of COPD, asthma bronchiale, allergic bronchitis, chronic bronchitis, chronic heart failure, nephritis, rheumatoide arthritis or emphysema.

In another aspect the invention relates to the use of a pharmaceutical composition comprising as a free combination (a) an effective amount of a PDE4 inhibitor and optionally a pharmaceutically acceptable carrier and (b) an effective amount of a PDE5 inhibitor and optionally a pharmaceutically acceptable carrier for the treatment of COPD, asthma bronchiale, allergic bronchitis, chronic bronchitis, chronic heart failure, nephritis, rheumatoide arthritis or emphysema.

### DETAILED DESCRIPTION OF THE INVENTION

The combination therapy, which is the subject matter of present invention, comprises administering a PDE4 inhibitor with a PDE5 inhibitor to prevent the onset of a disease in which phosphodiesterase 4 (PDE4) and/or phosphodiesterase 5 (PDE5) activity is detrimental.

The invention thus relates to the combined use of a PDE4 inhibitor and a PDE5 inhibitor in preventing the symptoms of, or treating a disease in which phosphodiesterase 4 (PDE4) and/or phosphodiesterase 5 (PDE5) activity is detrimental.

The PDE4 inhibitors useful in this invention may be any compound that is known to inhibit the PDE4 enzyme or which is discovered to act as a PDE4 inhibitor, and which is only or essentially only a PDE4 inhibitor, not compounds which inhibit to a degree of exhibiting a therapeutic effect other members of the PDE family as well as PDE4.

One group of PDE4 inhibitors that may be usefully employed in the present invention [hereinafter referred to as “SELECTED PDE4 INHIBITORs”] include a compound of formula (1)

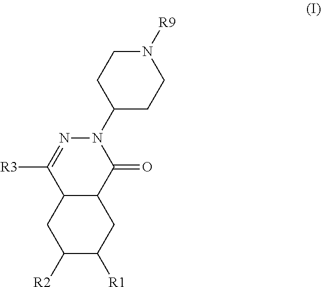

in which
R1 and R2 are both hydrogen or together form an additional bond,
R3 represents a benzene derivative of formula (a) or (b)

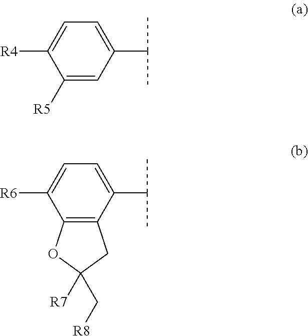

wherein
R4 is 1-4C-alkoxy or 1-4C-alkoxy which is completely or predominantly substituted by fluorine,
R5 is 1-8C-alkoxy, 3-7C-cycloalkoxy, 3-7C-cycloalkylmethoxy, or 1-4C-alkoxy which is completely or predominantly substituted by fluorine,
R6 is 1-4C-alkoxy, 3-5C-cycloalkoxy, 3-5C-cycloalkylmethoxy, or 1-4C-alkoxy which is completely or predominantly substituted by fluorine,
R7 is 1-4C-alkyl and
R8 is hydrogen or 1-4C-alkyl,
or wherein
R7 and R8 together and with inclusion of the two carbon atoms, to which they are bonded, form a spiro-linked 5-, 6- or 7-membered hydrocarbon ring, optionally interrupted by an oxygen or sulphur atom,
R9 is 1-4C-Alkyl, —S(O)₂—R10, —S(O)₂—(CH₂)ₙ—R11, —(CH₂)ₘ—S(O)₂—R12, —C(O)R13, —C(O)—(CH₂)ₙ—R14, —(CH₂)ₘ—C(O)—R15, Hetaryl, Aryl1 or Aryl2-(1-4C)-alkyl,
R10 is 1-4C-alkyl, 5-dimethylaminonaphthalin-1-yl, —N(R16)R17, phenyl or phenyl substituted by R18 and/or R19,
R11 is —N(R16)R17,
R12 is —N(R16)R17,
R13 is 1-4C-alkyl, hydroxycarbonyl-1-4C-alkyl, phenyl, pyridyl, 4-ethyl-piperazin-2,3-dion-1-yl or —N(R16)R17,
R14 is —N(R16)R17,
R15 is —N(R16)R17, phenyl, phenyl substituted by R18 and/or R19 and/or R20,
R16 and R17 are independent from each other hydrogen, 1-7C-alkyl, 3-7C-cycloalkyl, 3-7C-cycloalkylmethyl, phenyl or phenyl substituted by R18 and/or R19 and/or R20, or R16 and R17 together and with inclusion of the nitrogen atom to which they are bonded, form a 4-morpholinyl-, 1-pyrrolidinyl-, 1-piperidinyl-, 1-hexahydroazepino- or a 1-piperazinyl-ring of formula (c)

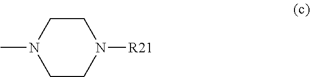

wherein
R21 is pyrid-4-yl, pyrid-4-ylmethyl, 1-4C-alkyl-dimethylamino, dimethylaminocarbonylmethyl, N-methyl-piperidin-4-yl, 4-morpholino-ethyl or tetrahydrofuran-2-ylmethyl,
R18 is halogen, nitro, cyano, carboxyl, 1-4C-alkyl, trifluoromethyl, 1-4C-alkoxy, 1-4C-alkoxycarbonyl, amino, mono- or di-1-4C-alkylamino, aminocarbonyl, 1-4C-alkylcarbonylamino or mono- or di-1-4C-alkylaminocarbonyl,
R19 is halogen, amino, nitro, 1-4C-alkyl or 1-4C-alkoxy,
R20 is halogen,
Hetaryl is pyrimidin-2-yl, thieno-[2,3-d]pyrimidin-4-yl, 1-methyl-1H-pyrazolo-[3,4-d]pyrimidin-4-yl, thiazolyl, imidazolyl or furanyl,
Aryl1 is pyridyl, phenyl or phenyl substituted by R18 and/or R19,
Aryl2 is pyridyl, phenyl, phenyl substituted by R18 and/or R19, 2-oxo-2H-chromen-7-yl or 4-(1,2,3-thiadiazol-4-yl)phenyl,
n is an integer from 1 to 4,
m is an integer from 1 to 4,
   or a pharmaceutically acceptable salt or a N-oxide thereof or a pharmaceutically acceptable salt of the latter.

1-4C-Alkyl is a straight-chain or branched alkyl radical having 1 to 4 carbon atoms. Examples are the butyl, isobutyl, sec-butyl, tert-butyl, propyl, isopropyl, ethyl and methyl radicals.

1-4C-Alkoxy is a radical which, in addition to the oxygen atom, contains a straight-chain or branched alkyl radical having 1 to 4 carbon atoms. Alkoxy radicals having 1 to 4 carbon atoms which may be mentioned in this context are, for example, the butoxy, isobutoxy, sec-butoxy, tert-butoxy, propoxy, isopropoxy, ethoxy and methoxy radicals.

1-8C-Alkoxy is a radical which, in addition to the oxygen atom, contains a straight-chain or branched alkyl radical having 1 to 8 carbon atoms. Alkoxy radicals having 1 to 8 carbon atoms which may be mentioned in this context are, for example, the octyloxy, heptyloxy, isoheptyloxy (5-methylhexyloxy), hexyloxy, isohexyloxy (4-methylpentyloxy), neohexyloxy (3,3-dimethylbutoxy), pentyloxy, isopentyloxy (3-methylbutoxy), neopentyloxy (2,2-dimethylpropoxy), butoxy, isobutoxy, sec-butoxy, tert-butoxy, propoxy, isopropoxy, ethoxy and methoxy radicals.

1-4C-Alkoxy which is completely or predominantly substituted by fluorine is, for example, the 2,2,3,3,3-pentafluoropropoxy, the perfluoroethoxy, the 1,2,2-trifluoroethoxy and in particular the 1,1,2,2-tetrafluoroethoxy, the 2,2,2-trifluoroethoxy, the trifluoromethoxy and the difluoromethoxy radical, of which the difluoromethoxy radical is preferred. “Predominantly” in this connection means that more than half of the hydrogen atoms of the 1-4C-alkoxy group are replaced by fluorine atoms.

3-7C-Cycloalkoxy stands for cyclopropyloxy, cyclobutyloxy, cyclopentyloxy, cyclohexyloxy or cycloheptyloxy, of which cyclopropyloxy, cyclobutyloxy and cyclopentyloxy are preferred.

3-7C-Cycloalkylmethoxy stands for cyclopropylmethoxy, cyclobutylmethoxy, cyclopentylmethoxy, cyclohexylmethoxy or cycloheptylmethoxy, of which cyclopropylmethoxy, cyclobutylmethoxy and cyclopentylmethoxy are preferred.

3-5C-Cycloalkoxy stands for cyclopropyloxy, cyclobutyloxy and cyclopentyloxy.

3-5C-Cycloalkylmethoxy stands for cyclopropylmethoxy, cyclobutylmethoxy and cyclopentylmethoxy.

Halogen within the meaning of the present invention is bromine, chlorine or fluorine.

As spiro-linked 5-, 6- or 7-membered hydrocarbon rings, optionally interrupted by an oxygen or sulphur atom, may be mentioned the cyclopentane, cyclohexane, cycloheptane, tetrahydrofuran, tetrahydropyran and the tetrahydrothiophen ring.

1-4C-Alkoxycarbonyl is a carbonyl group to which one of the abovementioned 1-4C-alkoxy radicals is bonded. Examples are the methoxycarbonyl [CH₃O—C(O)-] and the ethoxycarbonyl [CH₃CH₂O—C(O)-] radical.

An 1-4C-Alkylcarbonylamino radical is, for example, the propionylamino [C₃H₇C(O)NH-] and the acetylamino radical [CH₃C(O)NH-].

Mono- or Di-1-4C-alkylamino radicals contain in addition to the nitrogen atom, one or two of the abovementioned 1-4C-alkyl radicals. Preferred are the di-1-4C-alkylamino radicals, especially the dimethylamino, the diethylamino and the diisopropylamino radical.

Mono- or Di-1-4C-alkylaminocarbonyl radicals contain in addition to the carbonyl group one of the abovementioned mono- or di-1-4C-alkylamino radicals. Examples which may be mentioned are the N-methyl- the N,N-dimethyl-, the N-ethyl-, the N-propyl-, the N,N-diethyl- and the N-isopropylamino-carbonyl radical.

Salts encompassed within the term “pharmaceutically acceptable salts” refer to non-toxic salts of the compounds which are generally prepared by reacting a free base with a suitable organic or inorganic acid or by reacting the acid with a suitable organic or inorganic base. Particular mention may be made of the pharmaceutically acceptable inorganic and organic acids customarily used in pharmacy. Those suitable are in particular water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)-benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 1-hydroxy-2-naphthoic acid, the acids being employed in salt preparation—depending on whether it is a mono- or polybasic acid and depending on which salt is desired—in an equimolar quantitative ratio or one differing therefrom. As examples of salts with bases are mentioned the lithium, sodium, potassium, calcium, aluminium, magnesium, titanium, ammonium, meglumine or guanidinium salts, here, too, the bases being employed in salt preparation in an equimolar quantitative ratio or one differing therefrom.

It is understood that the active compounds and their pharmaceutically acceptable salts mentioned can also be present, for example, in the form of their pharmaceutically acceptable solvates, in particular in the form of their hydrates.

SELECTED PDE4 INHIBITORs, which are to be emphasized include a compound of formula (1), in which
R1 and R2 together form an additional bond,
R3 represents a benzene derivative of formula (a) or (b)

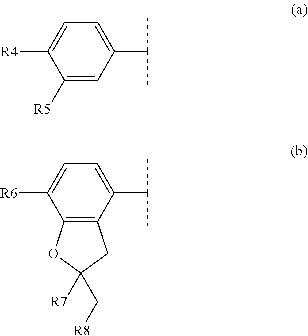

wherein
R4 is 1-4C-alkoxy,
R5 is 1-4C-alkoxy,
R6 is 1-2C-alkoxy,
R7 is methyl and
R8 is hydrogen,
R9 is 1-4C-alkyl, —S(O)₂—R10, —C(O)R13, —C(O)—(CH₂)ₙ—R14, —(CH₂)ₘ—C(O)—R15, Hetaryl, Aryl1 or Aryl2-(1-2C-)alkyl,
R10 is 1-4C-alkyl, 5-dimethylaminonaphthalin-1-yl, phenyl or phenyl substituted by R18,
R13 is 1-4C-alkyl, hydroxycarbonyl-1-4C-alkyl, pyridyl, 4-ethyl-piperazin-2,3-dion-1-yl or —N(R16)R17,
R14 is —N(R16)R17,
R15 is —N(R16)R17, phenyl or phenyl substituted by R18 and/or R19 and/or R20,
R16 and R17 are independent from each other hydrogen, 1-4C-alkyl, phenyl or phenyl substituted by R18 and/or R19 and/or R20, or R16 and R17 together and with inclusion of the nitrogen atom to which they are bonded, form a 4-morpholinyl ring or a 1-piperazinyl ring of formula (c)

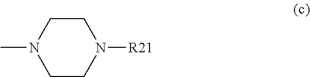

wherein
R21 is dimethylamino-1-4C-alkyl,
R18 is halogen, nitro, 1-4C-alkyl or 1-4C-alkoxycarbonyl,
R19 is amino,
R20 is halogen,
Hetaryl is pyrimidin-2-yl, thieno-[2,3-d]pyrimidin-4-yl or 1-methyl-1H-pyrazolo-[3,4-d]pyrimidin-4-yl,
Aryl1 is phenyl or phenyl substituted by R18,
Aryl2 is pyridyl, phenyl, 2-oxo-2H-chromen-7-yl or 4-(1,2,3-thiadiazol-4-yl)phenyl,
n is 1 or 2,
m is 1 or 2,
   or a pharmaceutically acceptable salt or a N-oxide thereof or a pharmaceutically acceptable salt of the latter.

SELECTED PDE4 INHIBITORs, which are preferred include a compound selected from
(4aS,8aR)-4-(3,4-Diethoxyphenyl)-2-[1-(toluene-4-sulfonyl)-piperidin-4-yl]-4-a,5,8,8a-tetrahydro-2H-phthalazin-1-one,
(4aS,8aR)-4-(3,4-Diethoxyphenyl)-2-(1-methanesulfonyl-piperidin-4-yl)-4-a,5,8,8a-tetrahydro-2H-phthalazin-1-one,
(4aS,8aR)-2-(1-Acetyl-piperidin-4-yl)-4-(3,4-diethoxyphenyl)-4-a,5,8,8a-tetrahydro-2H-phthalazin-1-one,
5-{4-[(4aS,8aR)-4-(3,4-Diethoxy-phenyl)-1-oxo-4-a,5,8,8a-tetrahydro-1H-phthalazin-2-yl]-piperidin-1-yl}-5-oxo-pentanoic acid,
(4aS,8aR)-4-(3,4-Diethoxyphenyl)-2-[1-(1-pyridin-4-yl-methanoyl)-piperidin-4-yl]-4-a,5,8,8a-tetrahydro-2H-phthalazin-1-one,
4-[(4aS,8aR)-4-(3,4-Diethoxyphenyl)-1-oxo-4-a,5,8,8a-tetrahydro-1H-phthalazin-2-yl]-piperidine-1-carboxylic acid tert-butylamide,
4-[(4aS,8aR)-4-(3,4-Diethoxyphenyl)-1-oxo-4-a,5,8,8a-tetrahydro-1H-phthalazin-2-yl]-piperidine-1-carboxylic acid phenylamide,
4-[(4aS,8aR)-4-(3,4-Dimethoxyphenyl)-1-oxo-4-a,5,8,8a-tetrahydro-1H-phthalazin-2-yl]-piperidine-1-carboxylic acid tert-butylamide,
(cis)-4-[4-(7-Methoxy-2,2-dimethyl-2,3-dihydro-benzofuran-4-yl)-1-oxo-4-a,5,8,8a-tetrahydro-1H-phthalazin-2-yl]-piperidine-1-carboxylic acid tert-butylamide,
(4aS,8aR)-4-(3,4-Dimethoxyphenyl)-2-[1-(5-dimethylamino-naphthalene-1-sulfonyl)-piperidin-4-yl]-4-a,5,8,8a-tetrahydro-2H-phthalazin-1-one,
(4aS,8aR)-4-(3,4-Dimethoxyphenyl)-2-[1-(4-nitro-phenyl)-piperidin-4-yl]-4-a,5,8,8a-tetrahydro-2H-phthalazin-1-one,
(4aS,8aR)-4-(3,4-Dimethoxyphenyl)-2-(1-pyridin-4-ylmethyl-piperidin-4-yl)-4-a,5,8,8a-tetrahydro-2H-phthalazin-1-one,
(4aS,8aR)-4-(3,4-Dimethoxyphenyl)-2-[1-(morpholine-4-carbonyl)-piperidin-4-yl]-4-a,5,8,8a-tetrahydro-2H-phthalazin-1-one,
(4aS,8aR)-2-{1-[2-(4-Amino-3,5-dichloro-phenyl)-2-oxo-ethyl]-piperidin-4-yl}-4-(3,4-dimethoxy-phenyl)-4-a,5,8,8a-tetrahydro-2H-phthalazin-1-one,
4-(3,4-Dimethoxyphenyl)-21′-(1-methyl-1H-pyrazolo[3,4-d]pyrimidin-4-yl)-piperidin-4-yl]-4-a,5,8,8a-tetrahydro-2H-naphthalen-1-one,
(4aS,8aR)-4-(3,4-Dimethoxyphenyl)-2-(1-thieno[2,3-d]pyrimidin-4-yl-piperidin-4-yl)-4-a,5,8,8a-tetrahydro-2H-phthalazin-1-one,
(4aS,8aR)-4-(3,4-Dimethoxyphenyl)-2-(1-pyrimidin-2-yl-piperidin-4-yl)-4-a,5,8,8a-tetrahydro-2H-phthalazin-1-one,
(4aS,8aR)-4-(3,4-Dimethoxyphenyl)-2-[1-(2-oxo-2H-chromen-7-ylmethyl)-piperidin-4-yl]-4-a,5,8,8a-tetrahydro-2H-phthalazin-1-one,
(4aS,8aR)-4-(3,4-Dimethoxyphenyl)-2-[1-(2-morpholin-4-yl-2-oxo-ethyl)-piperidin-4-yl]-4-a,5,8,8a-tetrahydro-2H-phthalazin-1-one,
(4aS,8aR)-4-(3,4-Dimethoxyphenyl)-2-(1-phenethyl-piperidin-4-yl)-4-a,5,8,8a-tetrahydro-2H-phthalazin-1-one,
(4aS,8aR)-4-(3,4-Diethoxyphenyl)-2-[1-(morpholine-4-carbonyl)-piperidin-4-yl]-4-a,5,8,8a-tetrahydro-2H-phthalazin-1-one,
(4aS,8aR)-4-(3,4-Dimethoxyphenyl)-2-(1-pyridin-3-ylmethyl-piperidin-4-yl)-4-a,5,8,8a-tetrahydro-2H-phthalazin-1-one,
(4aS,8aR)-4-(3,4-Dimethoxy-phenyl)-2-(1-pyridin-2-ylmethyl-piperidin-4-yl)-4-a,5,8,8a-tetrahydro-2H-phthalazin-1-one,
(4aS,8aR)-4-(3,4-Diethoxyphenyl)-2-[1-(2-morpholin-4-yl-ethanoyl)-piperidin-4-yl]-4-a,5,8,8a-tetrahydro-2H-phthalazin-1-one,
(4aS,8aR)-4-(3,4-Diethoxyphenyl)-2-(1-{2-[4-(2-dimethylamino-ethyl)-piperazin-1-yl]-ethanoyl}-piperidin-4-yl)-4-a,5,8,8a-tetrahydro-2H-phthalazin-1-one,
2-{4-[(4aS,8aR)-4-(3,4-Dimethoxyphenyl)-1-oxo-4-a,5,8,8a-tetrahydro-1H-phthalazin-2-yl]-piperidin-1-yl}-N-Isopropyl-acetamide,
(4aS,8aR)-4-(3,4-Dimethoxyphenyl)-2-[1-(4-1,2,3-thiadiazol-4-yl-benzyl)-piperidin-4-yl]-4-a,5,8,8a-tetrahydro-2H-phthalazin-1-one,
1-(1-{4-[(4aS,8aR)-4-(3,4-Dimethoxyphenyl)-1-oxo-4-a,5,8,8a-tetrahydro-1H-phthalazin-2-yl]-piperidin-1-yl}-methanoyl)-4-ethyl-piperazine-2,3-dione,
4-(2-{4-[(4aS,8aR)-4-(3,4-Dimethoxy-phenyl)-1-oxo-4-a,5,8,8a-tetrahydro-1H-phthalazin-2-yl]-piperidin-1-yl}-ethanoylamino)-benzoic acid ethyl ester,
2-{4-[(4aS,8aR)-4-(3,4-Dimethoxyphenyl)-1-oxo-4-a,5,8,8a-tetrahydro-1H-phthalazin-2-yl]-piperidin-1-yl}-acetamide,
   or a pharmaceutically acceptable salt or a N-oxide thereof or a pharmaceutically acceptable salt of the latter.

SELECTED PDE4 INHIBITORs, which are particularly preferred include a compound selected from
(4aS,8aR)-4-(3,4-Dimethoxyphenyl)-2-(1-pyrimidin-2-yl-piperidin-4-yl)-4-a,5,8,8a-tetrahydro-2H-phthalazin-1-one,
(4aS,8aR)-4-(3,4-Dimethoxy-phenyl)-2-(1-pyridin-2-ylmethyl-piperidin-4-yl)-4-a,5,8,8a-tetrahydro-2H-phthalazin-1-one,
2-{4-[(4aS,8aR)-4-(3,4-Dimethoxyphenyl)-1-oxo-4-a,5,8,8a-tetrahydro-1H-phthalazin-2-yl]-piperidin-1-yl}-acetamide,
   or a pharmaceutically acceptable salt or a N-oxide thereof or a pharmaceutically acceptable salt of the latter.

The preparation of the SELECTED PDE4 INHIBITORs as well as their use as phosphodiesterase (PDE) 4 inhibitors is described in WO02/064584.

Another group of PDE4 inhibitors that may be usefully employed in the present invention include a compound selected from
N-(3,5-dichloropyrid-4-yl)-3-cyclopentyloxy-4-methoxybenzamide [INN: PICLAMILAST] and its salts; the preparation of this compound and its pharmaceutically acceptable salts as well as their use as PDE4 inhibitors is disclosed in the international patent application WO92/12961
3-[3-(cyclopentyloxy)-4-methoxybenzyl]-6-(ethylamino)-8-isopropyl-3H-purine [Research Code: V-11294A]; the preparation of this compound and its pharmaceutically acceptable salts as well as their use as PDE4 inhibitors is disclosed in the international patent application WO95/00516
N-[9-methyl-4-oxo-1-phenyl-3,4,6,7-tetrahydropyrrolo[3,2,1-jk][1,4]benzo-diazepin-3(R)-yl]pyridine-4-carboxamide [Research Code: CI-1018]; the preparation of this compound and its pharmaceutically acceptable salts as well as their use as PDE4 inhibitors is disclosed in the international patent application WO96/11690.
3,7-dihydro-3-(4-chlorophenyl)-1-propyl-1H-purine-2,6-dione [INN AROFYLLINE]; the preparation of this compound and its pharmaceutically acceptable salts as well as their use as PDE4 inhibitors is disclosed in the European patent application EP0435811.
md N-(3,5-dichloro-4-pyridinyl)-2-[1-(4-fluorobenzyl)-5-hydroxy-1H-indol-3-yl]-2-oxoacetamide [Research Code: AWD-12-281]; the preparation of this compound and its pharmaceutically acceptable salts as well as their use as PDE4 inhibitors is disclosed in the international patent application WO98/09946
N-(3,5-dichloropyridin-4-yl)-2-[5-fluoro-1-(4-fluorobenzyl)-1H-indol-3-yl]-2-oxoacetamide [Research Code: AWD-12-343]; the preparation of this compound and its pharmaceutically acceptable salts as well as their use as PDE4 inhibitors is disclosed in the international patent application WO98/09946.
Tetrahydro-5-[4-methoxy-3-[(1S,2S,4R)-2-norbornyloxy]phenyl]-2(1H)-pyrimidone [INN: ATIZORAM]; the preparation of this compound and its pharmaceutically acceptable salts as well as their use as PDE4 inhibitors is disclosed in the European patent application EP0389282.
β-[3-(cyclopentyloxy)-4-methoxyphenyl]-1,3-dihydro-1,3-dioxo-2H-isoindole-2-propanamide [Research Code: CDC-801]; the preparation of this compound and its pharmaceutically acceptable salts as well as their use as PDE4 inhibitors is disclosed in the international patent application WO97/23457.
Methanesulfonic acid 2-(2,4-dichlorophenylcarbonyl)-3-ureidobenzo-furan-6-yl ester [INN: LIRIMILAST]; the preparation of this compound and its pharmaceutically acceptable salts as well as their use as PDE4 inhibitors is disclosed in the European patent application EP0731099.
3,5-dichloro-4-[8-methoxy-2-(trifluoromethyl)quinolin-5-ylcarbox-amido]pyridine-1-oxide [Research Code: SCH-351591]; the preparation of this compound and its pharmaceutically acceptable salts as well as their use as PDE4 inhibitors is disclosed in the international patent application WO00/26208;
cis-4-cyano-4-[3-cyclopentyloxy-4-methoxyphenyl]cyclohexane-1-carboxylic acid [INN: Cilomilast], the preparation of this compound and its pharmaceutically acceptable salts as well as their use as PDE4 inhibitors is disclosed in the international patent application WO93/19749
3-Cyclopropylmethoxy-4-difluoromethoxy-N-(3,5-dichloropyrid-4-yl)-benzamide [INN: ROFLUMILAST] as well as its N-oxide [3-cyclopropylmethoxy-4-difluoromethoxy-N-(3,5-dichloro-1-oxypyrid-4-yl)benzamide, the preparation of these compounds and their pharmaceutically acceptable salts as well as their use as PDE4 inhibitors is disclosed in the international patent application WO95/01338
the compounds with the research codes CDC-998, IC-485, CC-1088 and KW4490
   and the pharmaceutically acceptable salts of the above listed compounds. In this second group of PDE4 inhibitors, Roflumilast, Roflumilast-N-Oxide, Cilomilast and AWD-12-281 are particularly preferred.

The PDE5 inhibitors useful in this invention may be any compound that is known to inhibit the PDE5 enzyme or which is discovered to act as a PDE5 inhibitor, and which is only or essentially only a PDE5 inhibitor, not compounds which inhibit to a degree of exhibiting a therapeutic effect other members of the PDE family as well as PDE5.

A group of PDE5 inhibitors that may be usefully employed in the present invention include a compound selected from
SY-39: 4-Methyl-5-(4-pyridinyl)thiazole-2-carboxamide, the preparation of these compounds and their pharmaceutically acceptable salts as well as their use as PDE5 inhibitors is disclosed in the patent application EP0199968;
DIPYRIDAMOL: 2,2′,2″,2″-[(4,8-dipiperidinopyrimido[5,4-d]pyrimidine-2,6-diyl)-dinitrilo]-tetraethanol, the preparation of these compounds and their pharmaceutically acceptable salts as well as their use as PDE5 inhibitors is disclosed in the patent application DE1116676;
SKF-96231: 2-(2-propoxyphenyl)purin-6(1H)-one2-(2-propoxyphenyl)-1,7-dihydro-5H-purin-6-one, the preparation of these compounds and their pharmaceutically acceptable salts as well as their use as PDE5 inhibitors is disclosed in the patent application EP0293063;
ER-21355: 116-chloro-4-(3,4-methylenedioxybenzylamino)quinazolin-2-yl]-piperidine-4-carboxylic acid, the preparation of these compounds and their pharmaceutically acceptable salts as well as their use as PDE5 inhibitors is disclosed in the patent application WO9307124;
SCH-51866: (+)-cis-5-methyl-2-[4-(trifluoromethyl)benzyl]-3,4,5,6a,7,8,9-octahydrocyclopent[4,5]imidazo[2,1-b]purin-4-one, the preparation of these compounds and their pharmaceutically acceptable salts as well as their use as PDE5 inhibitors is disclosed in the patent application WO9419351;
A-02131-1: 5-[6-fluoro-1-(phenylmethyl)-1H-indazol-3-yl]-2-furan-methanol, the preparation of these compounds and their pharmaceutically acceptable salts as well as their use as PDE5 inhibitors is disclosed in the patent application EP0667345;
SCH-59498: cis-2-hexyl-5-methyl-3,4,5,6a,7,8,9,9a-octahydrocyclopent[4,5]imidazo-[2,1-b]purin-4-one, the preparation of these compounds and their pharmaceutically acceptable salts as well as their use as PDE5 inhibitors is disclosed in the patent application WO9119717;
E-4010: 4-(3-chloro-4-methoxybenzylamino)-1-(4-hydroxypiperidin-1-yl)-phthalazine-6-carbonitrile, the preparation of these compounds and their pharmaceutically acceptable salts as well as their use as PDE5 inhibitors is disclosed in the patent application WO9605176;
TADALAFIL: (6R,12aR)-2,3,6,7,12,12a-hexahydro-2-methyl-6-(3,4-methylenedioxy-phenyl)-pyrazino[2′,′: 6,1]pyrido[3,4-b]indole-1,4-dione, the preparation of these compounds and their pharmaceutically acceptable salts as well as their use as PDE5 inhibitors is disclosed in the patent application WO9519978;
VARDENAFIL: 2-[2-ethoxy-5-(4-ethylpiperazin-1-ylsulfonyl)phenyl]-5-methyl-7-propylimidazo[5,1-f][1,2,4]triazin-4(3H)-one, the preparation of these compounds and their pharmaceutically acceptable salts as well as their use as PDE5 inhibitors is disclosed in the patent application WO9924433;
UK-343664: 1-ethyl-4-[[3-[3-ethyl-4,7-dihydro-7-oxo-2-(2-pyridinylmethyl)-2H-pyrazolo[4,3-d]pyrimidin-5-yl]-4-propoxyphenyl]sulfonyl]-piperazine, the preparation of these compounds and their pharmaceutically acceptable salts as well as their use as PDE5 inhibitors is disclosed in the patent application WO9849166;
T-0156: 2-(2-methylpyridin-4-ylmethyl)-1-oxo-8-(2-pyrimidinylmethoxy)-4-(3,4,5-trimethoxyphenyl)-1,2-dihydro[2,7]naphthyridine-3-carboxylic acid methyl ester, the preparation of these compounds and their pharmaceutically acceptable salts as well as their use as PDE5 inhibitors is disclosed in the patent application WO0012503;
DA-8159: 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide, the preparation of these compounds and their pharmaceutically acceptable salts as well as their use as PDE5 inhibitors is disclosed in the patent application WO0027848;
FR-181074: 1-(2-chlorobenzyl)-3-isobutyryl-2-propylindole-6-carboxamide, the preparation of these compounds and their pharmaceutically acceptable salts as well as their use as PDE5 inhibitors is disclosed in the patent application WO9632379;
FR-226807: N-(3,4-dimethoxybenzyl)-2-[2-hydroxy-1(R)-methylethylamino]-5-nitrobenzamide, the preparation of these compounds and their pharmaceutically acceptable salts as well as their use as PDE5 inhibitors is disclosed in the patent application WO9954284;
SILDENAFIL: 5-[2-ethoxy-5-(4-methyl-1-piperazinylsulfonyl)phenyl]-1-methyl-3-n-propyl-1,6-dihydro-7H-pyrazolo[4,3-d]pyrimidin-7-one, the preparation of these compounds and their pharmaceutically acceptable salts as well as their use as PDE5 inhibitors is disclosed in the patent application EP0463756;
KF-31327: 3-ethyl-8-[2-[4-(hydroxymethyl)piperidin-1-yl]benzylamino]-2,3-dihydro-1H-imidazo[4,5-g]quinazoline-2-thione, the preparation of these compounds and their pharmaceutically acceptable salts as well as their use as PDE5 inhibitors is disclosed in the patent application WO9808848;
T-1032: 2-(4-aminophenyl)-1-oxo-7-(2-pyridinylmethoxy)-4-(3,4,5-trimethoxyphenyl)-1,2-dihydroisoquinoline-3-carboxylic acid methyl ester, the preparation of these compounds and their pharmaceutically acceptable salts as well as their use as PDE5 inhibitors is disclosed in the patent application WO9838168;
FR-229934: pentane-1-sulfonic acid [1-[3-(3,4-dichloro-benzyl)-2-methyl-3H-benzoimidazol-5-yl]-methanoyl}-amide, the preparation of these compounds and their pharmaceutically acceptable salts as well as their use as PDE5 inhibitors is disclosed in the patent application WO9900373;
BMS-263504: 1-[[3-(7,8-dihydro-8-oxo-1H-imidazo[4,5-g]quinazolin-6-yl)-4-propoxyphenyl]sulfonyl]-4-methylpiperazine, the preparation of these compounds and their pharmaceutically acceptable salts as well as their use as PDE5 inhibitors is disclosed in the patent application WO9964004;
WIN-65579: 1-cyclopentyl-6-(3-ethoxy-4-pyridinyl)-3-ethyl-1,7-dihydro-4H-pyrazolo[3,4-d]pyrimidin-4-one, the preparation of these compounds and their pharmaceutically acceptable salts as well as their use as PDE5 inhibitors is disclosed in the U.S. Pat. No. 5,294,612;
UK-371800: 3-ethyl-5-[5-(4-ethylpiperazin-1-ylsulfonyl)-2-[2-methoxy-[(R)-methylethoxy]pyridin-3-yl]-2-methyl-6,7-dihydro-2H-pyrazolo[4,3-d]-pyrimidin-7-one, the preparation of these compounds and their pharmaceutically acceptable salts as well as their use as PDE5 inhibitors is disclosed in the patent application WO9954333;
BF/GP-385: 2-(1H-imidazol-1-yl)-6-methoxy-4-(2-methoxyethylamino)-quinazoline, the preparation of these compounds and their pharmaceutically acceptable salts as well as their use as PDE5 inhibitors is disclosed in the patent application EP0579496;
CP-248: (1Z)—N-benzyl-2-[6-fluoro-2-methyl-3-(3,4,5-trimethoxybenzylidene)-3H-inden-1-yl]-acetamide, the preparation of these compounds and their pharmaceutically acceptable salts as well as their use as PDE5 inhibitors is disclosed in the patent application WO9747303;
ZAPRINAST: 3,6-dihydro-5-(o-propoxyphenyl)-7H-s-triazolo[4,5-d]pyrimidin-7-one, the preparation of these compounds and their pharmaceutically acceptable salts as well as their use as PDE5 inhibitors is disclosed in the patent application DE2162096; and
VESNARINONE: 3,4-dihydro-6-[4-(3,4-dimethoxybenzoyl)-1-piperazinyl]-2(1H)-quinolinone, the preparation of these compounds and their pharmaceutically acceptable salts as well as their use as PDE5 inhibitors is disclosed in the patent application DE3142982.

One group of PDE5 inhibitors that are particularly preferred in the present invention [hereinafter referred to as “SELECTED PDE5 INHIBITORs”] include TADALAFIL, SILDENAFIL, VARDENAFIL, UK357903, E8010 and TA-1790 and the pharmaceutically acceptable salts of these compounds.

“Diseases in which phosphodiesterase 4 (PDE4) and/or phosphodiesterase 5 (PDE5) activity is detrimental” which may be mentioned are in particular acute and chronic disorders of varying origin with an inflammatory component, a mismatch component and/or a remodelling component. Diseases which may be mentioned as examples are COPD, asthma bronchiale, allergic bronchitis, chronic bronchitis, chronic heart failure, nephritis, rheumatoide arthritis and emphysema.

The phrase “a mismatch component” refers to the disease component characterized
1. by a more or less pronounced collapse of the alveolar gas exchange function resulting in hypoxaemia (deterioration in gas exchange with decrease in the oxygen content of the patient's blood), wasted perfusion (uneconomical perfusion of unventilated areas) and wasted ventilation (uneconomical ventilation of poorly perfused areas) and/or
2. by a more or less pronounced collapse in perfusion of skeletal muscles resulting in wasted perfusion of unstressed muscle groups to the detriment of perfusion of stressed muscle groups.

In accordance with present invention, the mismatch component of a disease leads to a limitation in the patient's performance due to a deficient oxygen supply to the muscles in combination with a “squandering” of cardiorespiratory reserves and thus results of a limitation on muscular performance. The clinical symptoms are a limitation on performance and exercise-dependent or permanent dyspnoea.

In accordance to present invention, regulation of the “perfusion/demand matching” in skeletal muscles takes place in analogy to the lung through local release of endogenous vasodilators (especially NO/cGMP). The demand-oriented perfusion is in favor of the stressed muscle groups (muscular selectivity), and within the muscle groups in favor of the specifically stressed fibre types (intramuscular selectivity). The type of stress, duration of stress and level of stress thus determine under physiological conditions the specific perfusion profiles in each case. Various inflammatory disorders (e.g. COPD) may lead to a perfusion/demand mismatch. The consequence is wasted perfusion of unstressed muscle groups to the detriment of perfusion of stressed muscle groups, with the result of a limitation on muscular performance.

The phrase “a remodelling component” refers to structural changes in the morphology of the airways based on growth factor-induced proliferation of smooth muscle cells and fibroblasts in the airway epithelium and/or vasculature leading to a hyperplasia.

The phrase “combined use” (or “combination”) embraces the administration of a PDE4 inhibitor and a PDE5 inhibitor as part of a specific treatment regimen intended to provide a beneficial effect from the co-action of these therapeutic agents. Administration of these therapeutic agents in combination typically is carried out over a defined time period (usually minutes, hours, days or weeks depending upon the combination selected). “Combined use” generally is not intended to encompass the administration of two of these therapeutic agents as part of separate monotherapy regimens that incidentally and arbitrarily result in the combinations of the present invention.

“Combined use” or “combination” within the meaning of the present invention is to be understood as meaning that the individual components can be administered simultaneously (in the form of a combination medicament—“fixed combination”) or more or less simultaneously, or respectively in succession (from separate pack units—“free combination”; directly in succession or else alternatively at a relatively large time interval). As an example, one therapeutic agent could be taken in the morning and one later in the day. Or in another scenario, one therapeutic agent could be taken once daily and the other twice weekly. It is understood, that if individual components are administered directly in succession, the delay in administering the second component should not be such as to lose the beneficial therapeutic effect of the combination.

It is to be understood that present invention covers all combinations of particular and preferred aspects of the invention described herein. Thus, present invention clearly refers to all compounds mentioned herein as examples of a PDE4 inhibitor and a PDE5 inhibitor and to all possible consequential combinations. In particular, combinations which may be mentioned as preferred examples of a combination of a PDE4 inhibitor and a PDE5 inhibitor are
a combination of Sildenafil and Roflumilast,
a combination of Sildenafil and Cilomilast,
a combination of Tadalafil and Roflumilast,
a combination of Tadalafil and Cilomilast,
a combination of Vardenafil and Roflumilast, and
a combination of Vardenafil and Cilomilast.

Simultaneous administration can be effected by any appropriate route and, preferably, is accomplished, for example, by administering the therapeutic agents to the subject in need thereof by the oral route, or the intravenous route, or the intramuscular route, or by subcutaneous injection whereby the administration form has a fixed ratio of each therapeutic agent.

More or less simultaneous administration or administration in succession of each therapeutic agent can be effected by any appropriate route, including, but not limited to, oral routes, intravenous routes, intramuscular routes, and direct absorption through mucous membrane tissues. The therapeutic agents can be administered by the same route or by different routes. For example, both therapeutic agents of the combination may be administered by orally. In another example, a first therapeutic agent of the combination selected may be administered by intravenous or subcutaneous injection while the other therapeutic agent of the combination may be administered orally. The sequence in which the therapeutic agents are administered is not narrowly critical.

The most preferred route of administration of a PDE4 inhibitor is the oral route. In another preferred embodiment the PDE4 inhibitor administered by intravenous infusion or injection. In a further embodiment the PDE4 inhibitor is administered by intramuscular or subcutaneous injection. Other routes of administration are also contemplated, including intranasal and transdermal routes, and by inhalation.

The most preferred route of administration of a PDE5 inhibitor is the oral route. In another preferred embodiment the PDE5 inhibitor administered by intravenous infusion or injection. In a further embodiment the PDE5 inhibitor is administered by intramuscular or subcutaneous injection. Other routes of administration are also contemplated, including intranasal and transdermal routes, and by inhalation.

The therapeutic agent(s) of the present invention can be administered by a variety of methods known in the art, although for many therapeutic applications, the preferred route of administration for a fixed combination of a PDE4 inhibitor and a PDE5 inhibitor according to the invention is the oral route. The preferred route of administration for a free combination of a PDE4 inhibitor and a PDE5 inhibitor according to present invention is the oral route.

In case of pharmaceutical compositions, which are intended for oral administration, the therapeutic agent(s) are formulated to give medicaments according to processes known per se and familiar to the person skilled in the art. The therapeutic agents are employed as medicament, preferably in combination with suitable pharmaceutical carrier, in the form of tablets, coated tablets, capsules, caplets, emulsions, suspensions, syrups or solutions, the therapeutic agent content advantageously being between 0.1 and 95% by weight and, by the appropriate choice of the carrier, it being possible to achieve a pharmaceutical administration form precisely tailored to the therapeutic agent(s) and/or to the desired onset of action (e.g. a sustained-release form or an enteric form).

The person skilled in the art is familiar on the basis of his/her expert knowledge which carriers or excipients are suitable for the desired pharmaceutical formulations. In addition to solvents, gel-forming agents, tablet excipients and other active compound carriers, it is possible to use, for example, antioxidants, dispersants, emulsifiers, antifoams, flavor corrigents, preservatives, solubilizers, colorants or permeation promoters and complexing agents (e.g. cyclodextrins).

Suitable oral dosage forms of Roflumilast are described in the international patent application WO03/070279.

The therapeutic agent(s) are dosed in an order of magnitude customary for the individual dose. It is more likely possible that the individual actions of the therapeutic agents are mutually positively influenced and reinforced and thus the respective doses on the combined administration of the therapeutic agent(s) may be reduced compared with the norm.

In case of oral, intravenous or subcutaneous administration of a PDE4 inhibitor, the daily dose will likely be in the range from 0.001 to 3 mg/kg body weight of the subject to be treated, preferably by once daily administration.

In case of oral administration of the SELECTED PDE4 I NHIBITORs (4aS,8aR)-4-(3,4-Dimethoxyphenyl)-2-(1-pyrimidin-2-yl-piperidin-4-yl)-4-a,5,8,8a-tetrahydro-2H-phthalazin-1-one, (4aS,8aR)-4-(3,4-Dimethoxy-phenyl)-2-(1-pyridin-2-ylmethyl-piperidin-4-yl)-4-a,5,8,8a-tetrahydro-2H-phthalazin-1-one or 2-{4-[(4aS,8aR)-4-(3,4-Dimethoxyphenyl)-1-oxo-4-a,5,8,8a-tetrahydro-1H-phthalazin-2-yl]-piperidin-1-yl}-acetamide the adult daily dose is between 0.1 and 10 mg, preferably between 0.5 and 2 mg.

In case of oral administration of 3-cyclopropylmethoxy-4-difluoromethoxy-N-(3,5-dichloropyrid-4-yl)-benzamide (ROFLUMILAST), the adult daily dose is in the range from 50-1000 μg, preferably in the range from 250-500 μg, preferably by once daily administration.

In case of oral, intravenous or subcutaneous administration of a PDE5 inhibitor, the daily'dose will likely be in the range from 0.001 to 3 mg/kg body weight of the subject to be treated, preferably by once daily administration.

Tablet formulations for sildenafil, tadalafil and vardenafil are commercially available under the tradenames Viagra®, Cialis® and Levitra® respectively.

Commercially available tablet formulations for sildenafil contain 25, 50 or 100 mg of sildenafil. According to the Summary of Product Characteristics for Sildenafil, as a monotherapy the PDE5 inhibitor Sildenafil is generally administered orally to adults in a daily dose of 25, 50 or 100 mg.

Commercially available tablet formulations for vardenafil contain 5, 10 or 20 mg of vardenafil. According to the Summary of Product Characteristics for Vardenafil, as a monotherapy the PDE5 inhibitor Vardenafil is generally administered orally to adults in a daily dose of 5, 10 or 20 mg.

Commercially available tablet formulations for tadalafil contain 10 or 20 mg of tadalafil. According to the Summary of Product Characteristics for Tadalafil, as a monotherapy the PDE5 inhibitor Tadalafil is generally administered orally to adults in a daily dose of 10 or 20 mg.

### EXAMPLES

### Administration of the Combination

### Example 1

Patients suffering from COPD as defined by the “Global Initiative for chronic obstructive lung disease” (Pauwels R. A., et al., Global strategy for the diagnosis, management, and prevention of chronic obstructive pulmonary disease. NHLBI/WHO Global Initiative for Chronic Obstructive Lung Disease (GOLD) Workshop summary. Am. J. Respir. Crit. Care Med. 2001; 163: 1256-1276) are administered orally one tablet of Roflumilast (comprising 500 μg of Roflumilast) per day and once daily a tablet of Viagra (comprising 50 mg Sildenafil).

### Example 2

Patients suffering from COPD as defined by the “Global Initiative for chronic obstructive lung disease” (Pauwels R. A., et al., Global strategy for the diagnosis, management, and prevention of chronic obstructive pulmonary disease. NHLBI/WHO Global Initiative for Chronic Obstructive Lung Disease (GOLD) Workshop summary. Am. J. Respir. Crit. Care Med. 2001; 163: 1256-1276) are administered orally one tablet of Roflumilast (comprising 500 μg of Roflumilast) per day and each second day a tablet of Cialis (comprising 10 mg Tadalafil).

### Example 3

Patients suffering from COPD as defined by the “Global Initiative for chronic obstructive lung disease” (Pauwels R. A., et al., Global strategy for the diagnosis, management, and prevention of chronic obstructive pulmonary disease. NHLBI/WHO Global Initiative for Chronic Obstructive Lung Disease (GOLD) Workshop summary. Am. J. Respir. Crit. Care Med. 2001; 163: 1256-1276) are administered orally one tablet of Roflumilast (comprising 500 μg of Roflumilast) per day and once daily a tablet of Levitra (comprising 10 mg Vardenafil).

### Utility

Because of their PDE4- and PDE5-inhibitory properties, combinations of present invention are applicable in human and veterinary medicine, wherein—as an example—the combinations useful for preventing or reducing the onset of symptoms of a disease, or treating or reducing the severity of a disease in a patient in need thereof, in which disease phosphodiesterase 4 (PDE4) and/or phosphodiesterase 5 (PDE5) activity is detrimental. Said diseases are of varying origin and are characterized by an inflammatory component, a mismatch component and structural changes in the morphology of the airways (remodelling component). Thus, the combined use of a PDE4 inhibitor and a PDE5 inhibitor in accordance with present invention is applicable for preventing or reducing the onset of inflammation, or treating or reducing the severity of inflammation, mismatch and remodelling.

Surprisingly it has been found that the combined use of a PDE4- and a PDE5-inhibitor in the treatment of COPD, asthma bronchiale, allergic bronchitis, chronic bronchitis, chronic heart failure, nephritis, rheumatoide arthritis or emphysema is superior to a treatment with either a PDE4-inhibitor or a PDE5-inhibitor as the combined use of a PDE4- and a PDE5-inhibitor lead to a synergistic effect. This synergistic effect refers to an intensive mechanistic crosstalk between the pathomechanisms influenced by PDE4-inhibitors and those of PDE5-inhibitors. For example immune cells involved in COPD, which activity can be suppressed by treatment with PDE4-inhibitors, may release cytokines and growth factors, which induce and influence structural remodelling processes of vasculature. These remodelling processes are also under the control of PDE5-inhibitors, which are known to influence proliferation. Thus, combined use of a PDE4- and PDE5-inhibitor for the treatment of COPD, asthma bronchiale, allergic bronchitis, chronic bronchitis, chronic heart failure, nephritis, rheumatoide arthritis or emphysema is more effective than treatment with the individual inhibitors. In addition, treatment of COPD, asthma bronchiale, allergic bronchitis, chronic bronchitis, chronic heart failure, nephritis, rheumatoide arthritis or emphysema by use of a composition comprising a PDE4- and a PDE5-inhibitor allows the adaptation of a dose scheme of both inhibitors in order to get a useful ratio of plasma concentrations of the PDE4- and PDE5-inhibitor by considering the different pharmacokinetic behaviour of these drugs.

Pharmaceutical compositions of present invention may be prescribed to the patient in “patient pack” containing the whole course of treatment in a single package. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions and, therefore, lead generally to more successful treatment. It will be understood that the administration of the combination of present invention by means of a single patient pack, or patient packs of each component compound, and containing a package insert instructing the patient to the correct use of the invention is a desirable additional feature of the invention. Thus, the use of a pharmaceutical composition of present invention may help a patient (1) to prevent or reduce the onset of symptoms of a disease in which phosphodiesterase 4 (PDE4) and/or phosphodiesterase 5 (PDE5) activity is detrimental, or to treat or reduce the severity of a disease in which phosphodiesterase 4 (PDE4) and/or phosphodiesterase 5 (PDE5) activity is detrimental by use of one combination, and (2) to increase his compliance because of the use of a “patient pack”.

1.-24. (canceled)

25. A pharmaceutical composition comprising in a single dosage form
(a) an effective amount of a PDE4 inhibitor and
(b) an effective amount of a PDE5 inhibitor, and optionally
(c) a pharmaceutically acceptable carrier.

26. The pharmaceutical composition according to claim 25, wherein the single dosage form is an oral dosage form.

27. A pharmaceutical composition comprising
(a) in a first dosage form an effective amount of a PDE4 inhibitor and optionally a pharmaceutically acceptable carrier and
(b) in a second dosage form an effective amount of a PDE5 inhibitor and optionally a pharmaceutically acceptable carrier.

28.-46. (canceled)

47. The pharmaceutical composition according to claim 27, wherein the first and the second dosage forms are oral dosage forms.

48. The pharmaceutical composition according to claim 25, wherein the PDE4 inhibitor is 3-cyclopropylmethoxy-4-difluoromethoxy-N-(3,5-dichloropyrid-4-yl)-benzamide (Roflumilast) or a pharmaceutically acceptable salt, N-oxide, or a pharmaceutically acceptable salt of the N-oxide thereof and wherein the PDE5 inhibitor is selected from the group consisting of 5-[2-ethoxy-5-(4-methyl-1-piperazinylsulfonyl)phenyl]-1-methyl-3-n-propyl-1,6-dihydro-7H-pyrazolo[4,3-d]pyrimidin-7-one (SILDENAFIL), a pharmaceutically acceptable salt of SILDENAFIL, (6R,12aR)-2,3,6,7,12,12a-hexahydro-2-methyl-6-(3,4-methylenedioxy-phenyl)-pyrazino[2′,1′:6,1]pyrido[3,4-b]indole-1,4-dione (TADALAFIL), a pharmaceutically acceptable salt of TADALAFIL, 2-[2-ethoxy-5-(4-ethylpiperazin-1-ylsulfonyl)phenyl]-5-methyl-7-propylimidazo[5,1-f][1,2,4]triazin-4(3H)-one (VARDENAFIL) and a pharmaceutically acceptable salt of VARDENAFIL.

49. The pharmaceutical composition according to claim 48, wherein the PDE4 inhibitor is 3-cyclopropylmethoxy-4-difluoromethoxy-N-(3,5-dichloropyrid-4-yl)-benzamide (Roflumilast) and wherein the PDE5 inhibitor is selected from the group consisting of 5-[2-ethoxy-5-(4-methyl-1-piperazinylsulfonyl)phenyl]-1-methyl-3-n-propyl-1,6-dihydro-7H-pyrazolo[4,3-d]pyrimidin-7-one (SILDENAFIL) and a pharmaceutically acceptable salt of SILDENAFIL.

50. The pharmaceutical composition according to claim 26, wherein the PDE4 inhibitor is 3-cyclopropylmethoxy-4-difluoromethoxy-N-(3,5-dichloropyrid-4-yl)-benzamide (Roflumilast) or a pharmaceutically acceptable salt, N-oxide, or a pharmaceutically acceptable salt of the N-oxide thereof and wherein the PDE5 inhibitor is selected from the group consisting of 5-[2-ethoxy-5-(4-methyl-1-piperazinylsulfonyl)phenyl]-1-methyl-3-n-propyl-1,6-dihydro-7H-pyrazolo[4,3-d]pyrimidin-7-one (SILDENAFIL), a pharmaceutically acceptable salt of SILDENAFIL, (6R,12aR)-2,3,6,7,12,12a-hexahydro-2-methyl-6-(3,4-methylenedioxy-phenyl)-pyrazino[2′,1′:6,1]pyrido[3,4-b]indole-1,4-dione (TADALAFIL), a pharmaceutically acceptable salt of TADALAFIL, 2-[2-ethoxy-5-(4-ethylpiperazin-1-ylsulfonyl)phenyl]-5-methyl-7-propylimidazo[5,1-f][1,2,4]triazin-4(3H)-one (VARDENAFIL) and a pharmaceutically acceptable salt of VARDENAFIL.

51. The pharmaceutical composition according to claim 50, wherein the PDE4 inhibitor is 3-cyclopropylmethoxy-4-difluoromethoxy-N-(3,5-dichloropyrid-4-yl)-benzamide (Roflumilast) and wherein the PDE5 inhibitor is selected from the group consisting of 5-[2-ethoxy-5-(4-methyl-1-piperazinylsulfonyl)phenyl]-1-methyl-3-n-propyl-1,6-dihydro-7H-pyrazolo[4,3-d]pyrimidin-7-one (SILDENAFIL) and a pharmaceutically acceptable salt of SILDENAFIL.

52. The pharmaceutical composition according to claim 27, wherein the PDE4 inhibitor is 3-cyclopropylmethoxy-4-difluoromethoxy-N-(3,5-dichloropyrid-4-yl)-benzamide (Roflumilast) or a pharmaceutically acceptable salt, N-oxide, or a pharmaceutically acceptable salt of the N-oxide thereof and wherein the PDE5 inhibitor is selected from the group consisting of 5-[2-ethoxy-5-(4-methyl-1-piperazinylsulfonyl)phenyl]-1-methyl-3-n-propyl-1,6-dihydro-7H-pyrazolo[4,3-d]pyrimidin-7-one (SILDENAFIL), a pharmaceutically acceptable salt of SILDENAFIL, (6R,12aR)-2,3,6,7,12,12a-hexahydro-2-methyl-6-(3,4-methylenedioxy-phenyl)-pyrazino[2′,1′:6,1]pyrido[3,4-b]indole-1,4-dione (TADALAFIL), a pharmaceutically acceptable salt of TADALAFIL, 2-[2-ethoxy-5-(4-ethylpiperazin-1-ylsulfonyl)phenyl]-5-methyl-7-propylimidazo[5,1-f][1,2,4]triazin-4(3H)-one (VARDENAFIL) and a pharmaceutically acceptable salt of VARDENAFIL.

53. The pharmaceutical composition according to claim 52, wherein the PDE4 inhibitor is 3-cyclopropylmethoxy-4-difluoromethoxy-N-(3,5-dichloropyrid-4-yl)-benzamide (Roflumilast) and wherein the PDE5 inhibitor is selected from the group consisting of 5-[2-ethoxy-5-(4-methyl-1-piperazinylsulfonyl)phenyl]-1-methyl-3-n-propyl-1,6-dihydro-7H-pyrazolo[4,3-d]pyrimidin-7-one (SILDENAFIL) and a pharmaceutically acceptable salt of SILDENAFIL.

54. The pharmaceutical composition according to claim 47, wherein the PDE4 inhibitor is 3-cyclopropylmethoxy-4-difluoromethoxy-N-(3,5-dichloropyrid-4-yl)-benzamide (Roflumilast) or a pharmaceutically acceptable salt, N-oxide, or a pharmaceutically acceptable salt of the N-oxide thereof and wherein the PDE5 inhibitor is selected from the group consisting of 5-[2-ethoxy-5-(4-methyl-1-piperazinylsulfonyl)phenyl]-1-methyl-3-n-propyl-1,6-dihydro-7H-pyrazolo[4,3-d]pyrimidin-7-one (SILDENAFIL), a pharmaceutically acceptable salt of SILDENAFIL, (6R,12aR)-2,3,6,7,12,12a-hexahydro-2-methyl-6-(3,4-methylenedioxy-phenyl)-pyrazino[2′,1′:6,1]pyrido[3,4-b]indole-1,4-dione (TADALAFIL), a pharmaceutically acceptable salt of TADALAFIL, 2-[2-ethoxy-5-(4-ethylpiperazin-1-ylsulfonyl)phenyl]-5-methyl-7-propylimidazo[5,1-f][1,2,4]triazin-4(3H)-one (VARDENAFIL) and a pharmaceutically acceptable salt of VARDENAFIL.

55. The pharmaceutical composition according to claim 54, wherein the PDE4 inhibitor is 3-cyclopropylmethoxy-4-difluoromethoxy-N-(3,5-dichloropyrid-4-yl)-benzamide (Roflumilast) and wherein the PDE5 inhibitor is selected from the group consisting of 5-[2-ethoxy-5-(4-methyl-1-piperazinylsulfonyl)phenyl]-1-methyl-3-n-propyl-1,6-dihydro-7H-pyrazolo[4,3-d]pyrimidin-7-one (SILDENAFIL) and a pharmaceutically acceptable salt of SILDENAFIL.

56. A method of treating COPD in a patient comprising administering to a patient in need thereof the pharmaceutical composition according to claim 48.

57. A method of treating COPD in a patient comprising administering to a patient in need thereof the pharmaceutical composition according to claim 50.

58. A method of treating COPD in a patient comprising administering to a patient in need thereof the pharmaceutical composition according to claim 52.

59. A method of treating COPD in a patient comprising administering to a patient in need thereof the pharmaceutical composition according to claim 54.
